SEQUENCE LISTING (1 sequence)

SEQ ID NO: 1 - protein, 144 aa, Homo Sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

(19) United States
(12) Patent Application Publication
(10) Pub. No.: US 2015/0174204 A1
(43) Pub. Date: Jun. 25, 2015

(54) GRANULOCYTE-MACROPHAGE COLONY-STIMULATING FACTOR FOR THE TREATMENT OF BRONCHIAL ASTHMA

(71) Applicant: Reponex Pharmaceuticals ApS, Hørsholm (DK)

(72) Inventor: Lars Heslet, Gentofte (DK)

(73) Assignee: Reponex Pharmaceuticals Aps, Horsholm (DK)

(21) Appl. No.: 14/415,717

(22) PCT Filed: Jul. 19, 2013
(86) PCT No.: PCT/DK2013/050246

§ 371 Date: Jan. 19, 2015

Related U.S. Application Data
(60) Provisional application No. 61/673,795, filed on Jul. 20, 2012.

(51) Int. Cl. A61K 38/19

(52) U.S. Cl. CPC A61K 38/193

(57) ABSTRACT

The present invention relates to granulocyte-macrophage colony-stimulating factor (GM-CSF) for use in the treatment of bronchial asthma by administering via the airway an effective amount of GM-CSF or a functional homologue thereof. The invention further relates to a method for treating bronchial asthma comprising the administration of GM-CSF to a patient in need thereof, in particular moderate and severe forms of bronchial asthma.

All patent and non-patent references cited in this application or in the priority application are hereby incorporated by reference in their entirety.

### FIELD OF INVENTION

The present invention relates to granulocyte-macrophage colony-stimulating factor (GM-CSF) for use in the treatment of bronchial asthma. The invention further relates to a method of treating bronchial asthma comprising the administration of GM-CSF to a patient in need thereof.

### BACKGROUND OF INVENTION

Granulocyte-macrophage colony-stimulating factor, GM-CSF, was originally identified as a hemopoietic growth factor. Human GM-CSF stimulates the growth of myeloid and erythroid progenitors in vitro and activates monocytes, macrophages and granulocytes in several immune and inflammatory processes (Gasson et al., 1990b; Gasson et al., 1990a; Hart et al., 1988; Rapoport et al., 1992). It is produced by a number of cell types including lymphocytes, monocytes, endothelial cells, fibroblasts and some malignant cells (Metcalf 1986; Clark and Kamen, 1987; Hart et al., 1988). In addition to having a function of growth stimulation and differentiation on hemopoietic precursor cells, GM-CSF also was discovered as having a variety of effects on cells of the immune system expressing the GM-CSF receptor (for review, see Hamilton, 2002; de Groot et al., 1998).

Granulocyte-macrophage colony-stimulating factor inhalation therapy has been disclosed for treating patients suffering from idiopathic pulmonary alveolar proteinosis, a rare lung disease characterized by the accumulation of surfactant that fills the terminal airways and alveoli, thereby impairing respiratory function (Tazawa et al., 2006).

### SUMMARY OF INVENTION

In one aspect, the present invention relates to GM-CSF for use in the treatment of bronchial asthma. The GM-CSF may be administered to a subject in need thereof via the airway, e.g. by inhalation or intratracheal, intrabronchial or bronchoalveolar administration. Hence, the present invention further relates to a method for treating bronchial asthma comprising the administration of an effective amount of GM-CSF to a patient in need thereof.

### DETAILED DESCRIPTION OF THE INVENTION

The present invention relates to GM-CSF for use in the treatment of bronchial asthma. The GM-CSF may be administered in any suitable way known to a person of skill, particularly by inhalation of a nebulized solution of GM-CSF or of GM-CSF in powder form, or by any other appropriate means of intratracheal, intrabronchial or bronchoalveolar administration. The GM-CSF may be administered to human subjects including both adults and children. The GM-CSF may be purified or concentrated natural human GM-CSF or a functional homologue thereof, however prepared.

The GM-CSF may further be a recombinantly produced human GM-CSF. Thus the GM-CSF may be purified or concentrated natural human GM-CSF, or recombinantly produced human GM-CSF, or a functional homologue thereof, however prepared.

### Bronchial Asthma

Bronchial asthma is a common chronic inflammatory disease of the airways characterized by variable and recurring symptoms, reversible airflow obstruction, and bronchospasm. Symptoms include wheezing, coughing, chest tightness, and shortness of breath. Bronchial asthma is clinically classified according to the frequency of symptoms, forced expiratory volume in 1 second (FEV1), and peak expiratory flow rate. Bronchial asthma may also be classified as atopic (extrinsic) or non-atopic (intrinsic).

Bronchial asthma is thought to be caused by a combination of genetic and environmental factors. Treatment of acute symptoms is usually with an inhaled short-acting β₂-adrenergic receptor agonist (such as salbutamol). Symptoms can be prevented by avoiding triggers, such as allergens and irritants, and by inhaling corticosteroids. Leukotriene antagonists are less effective than corticosteroids and thus less preferred.

The diagnosis of bronchial asthma is usually made on the basis of the pattern of symptoms and signs and/or the response to therapy over time. The prevalence of bronchial asthma has increased significantly since the 1970s. As of 2010, 300 million people were affected worldwide. In 2009 bronchial asthma caused 250,000 deaths globally. Despite this, with proper control of bronchial asthma with step down therapy, prognosis is generally good.

Other means of control of bronchial asthma may also lead to a good prognosis.

Bronchial asthma is defined by the Global Initiative for Asthma as “a chronic inflammatory disorder of the airways in which many cells and cellular elements play a role. The chronic inflammation is associated with airway hyperresponsiveness that leads to recurrent episodes of wheezing, breathlessness, chest tightness and coughing particularly at night or in the early morning. These episodes are usually associated with widespread, but variable airflow obstruction within the lung that is often reversible either spontaneously or with treatment”.

Although bronchial asthma is a chronic obstructive condition, it is not considered as a part of chronic obstructive pulmonary disease as this term refers specifically to combinations of disease that are irreversible such as bronchiectasis, chronic bronchitis, and emphysema. Unlike these diseases, the airway obstruction in bronchial asthma is usually reversible; however, if left untreated, the chronic inflammation from bronchial asthma can lead the lungs to become irreversibly obstructed because of airways remodeling. In contrast to emphysema, bronchial asthma, as its name implies, affects the bronchi, not the alveoli.

### Clinical Classification of Severity of Bronchial Asthma

The clinical severity of asthma may be classified according to the Table 1 below.

TABLE 1

| Severity in | | | | |
|---|---|---|---|---|
| patients ≧12 | Symptom | Night-time | % FEV₁ of | FEV₁ |
| years of age | frequency | symptoms | predicted | variability |
| Intermittent | ≦2 per week | ≦2 per month | ≧80% | <20% |
| Mild | >2 per week | 3-4 per month | ≧80% | 20-30% |
| persistent | but not daily | | | |
| Moderate | Daily | >1 per week but | 60-80% | >30% |
| persistent | | not nightly | | |
| Severe | Throughout | Frequent (often | <60% | >30% |
| persistent | the day | 7x/week) | | |

The persistent forms of bronchial asthma may be classified as mild, moderate and severe.

The mild form is usually treated with β₂-agonists with good effect. The term β₂-agonists is a common denomination of β₂-adrenergic receptor agonists.

Moderate forms of asthma are usually treated successfully with a combination of β₂-agonists and steroids. More particularly, moderate forms of asthma are usually treated successfully with a combination of β₂-agonists and corticosteroids.

Severe forms of bronchial asthma do not show good clinical response to treatment with β₂-agonists and steroids only work slowly. In severe bronchial asthma, eosinophil granulocytes are thought to be involved in an adverse or negative process, presumably by secreting toxins that can lead to mucosal injury and plugging of the airways, thus resulting in the life-threatening clinical condition of acute severe bronchial asthma. The mucosal damage resulting in partial and/or complete plugging of the airways is hypothesized to be the reason why β₂-agonists essentially have no effect in severe bronchial asthma and why steroids (such as corticosteroids) only work slowly.

According to the present invention, GM-CSF may be used to treat all types of clinical classifications of bronchial asthma mentioned herein above, particularly persistent forms of bronchial asthma, such as severe bronchial asthma, which display a limited clinical response to currently used therapeutics.

In a particular embodiment, GM-CSF is used to treat acute severe bronchial asthma, either prophylactically or therapeutically. Patients suffering from severe bronchial asthma and those at risk of developing acute severe bronchial asthma may particularly benefit from treatment with GM-CSF.

Prophylaxis may be equivalent to reducing risk of the development of symptoms associated with bronchial asthma, such as airway hyperresponsiveness, recurrent episodes of wheezing, breathlessness, chest tightness, coughing particularly at night or in the early morning and widespread, but variable airflow obstruction within the lung.

GM-CSF is believed to exert its effect on macrophages in the small airways, which are thus stimulated to transform into dendritic cells which are able to clear the clogging of the airways associated with severe forms of bronchial asthma.

### GM-CSF

Colony-stimulating factors (CSFs) are glycoproteins that stimulate the growth of hematopoietic progenitors and enhance the functional activity of mature effector cells. In brief, at the level of immature cells, CSFs ensure the self-renewal of the staminal pool and activate the first stage of hematopoietic differentiation; in the middle stage, when cell proliferation is associated with a progressive acquisition of the characteristics of mature cells, they enormously enhance the number of differentiating cells; in the terminal stage they control the circulation and the activation of mature cells.

Mature GM-CSF is a monomeric protein of 127 amino acids with several potential glycosylation sites. The variable degree of glycosylation results in a molecular mass or weight range between 14 kDa and 35 kDa. Non-glycosylated and glycosylated GM-CSF show similar activity in vitro (Cebon et al., 1990). The crystallographic analysis of GM-CSF revealed a barrel-shaped structure composed of four short alpha helices (Diederichs et al., 1991). There are two known sequence variants of GM-CSF. The active form of the GM-CSF protein is found extracellularly as a homodimer in vivo.

GM-CSF exerts its biological activity by binding to its receptor. The most important sites of GM-CSF receptor (GM-CSF-R) expression are on the cell surface of myeloid cells, such as alveolar macrophages types I & II, epithelial pulmonary cells and endothelial cells, whereas lymphocytes are GM-CSF-R-negative. The native receptor is composed of at least two subunits, alpha and beta. The alpha subunit imparts ligand specificity and binds GM-CSF with nanomolar affinity (Gearing et al., 1989; Gasson et al., 1986). The beta subunit is also part of the interleukin-3 and interleukin-5 receptor complexes and, in association with the GM-CSF receptor alpha subunit and GM-CSF, leads to the formation of a complex with picomolar binding affinity (Hayashida et al., 1990). The binding domains on GM-CSF for the receptor have been mapped: GM-CSF interacts with the beta subunit of its receptor via a very restricted region in the first alpha helix of GM-CSF (Shanafelt et al., 1991a; Shanafelt et al., 1991b; Lopez et al., 1991). Binding to the alpha subunit could be mapped to the third alpha helix, helix C, the initial residues of the loop joining helices C and D, and to the carboxy-terminal tail of GM-CSF (Brown et al., 1994).

Formation of the GM-CSF trimeric receptor complex leads to the activation of complex signaling cascades involving molecules of the JAK/STAT families, She, Ras, Raf, the MAP kinases, phosphatidylinositol-3-kinase and NFκB, finally leading to transcription of c-myc, c-fos and c-jun. Activation is mainly induced by the beta subunit of the receptor (Hayashida et al., 1990; Kitamura et al., 1991; Sato et al., 1993). The shared beta subunit is also responsible for the overlapping functions exerted by IL-3, IL-5 and GM-CSF (for review see: de Groot et al., 1998).

Apart from its hemopoietic growth and differentiation stimulating activity, GM-CSF functions especially as a pro-inflammatory cytokine. Macrophages, e.g. alveolar macrophages types I & II and monocytes as well as neutrophils and eosinophils are activated by GM-CSF, resulting in the release of other cytokines and chemokines, matrix-degrading proteases, increased HLA expression and increased expression of cell adhesion molecules or receptors for CC-chemokines which in turn leads to increased chemotaxis of inflammatory cells into inflamed tissue.

As mentioned above, macrophages, eosinophils and basophils express GM-CSF receptors on their cell surface. The macrophages are on the air side of the air-blood barrier and the eosinophils are on the other side in the blood compartment at the level of the peripheral airways.

In order to treat or prevent bronchial asthma, the present invention may decrease the Tₕ2 immune response that is mediated by IgE antibodies, decrease activation of eosinophilic cells, and decrease eosinophilic toxin (ET). The present invention may further increase a Tₕ1 immune response by increasing cellular immunity and transforming the resting macrophages into dendritic cells. The increase in the Tₕ1 immune response may occur simultaneously with, or before or after the decrease in the Tₕ2 immune response.

Thus, without being bound by theory, it is believed that when GM-CSF or a functional homologue thereof is administered via the airways, the switch of the derailed Tₕ2 response to promotion of the Tₕ1 subset is caused by the sole stimulation of the GM-CSF receptors on cells on air side (for example the cells in or bordering the lumen) of the small airways. Subsequently dendritic cells orchestrate the immune defense in concert with a T helper cell inflammation.

GM-CSF is a protein of a considerable size, and therefore the transport of the protein across the air-blood barrier is minor and in some cases non-existent. Administration of GM-CSF into the airways may thus ensure that the majority of the GM-CSF reaches the cells in the airways (in the lumen of the bronchioles or alveoli), and not the cells in the blood or tissue compartments. Thus, without being bound by theory, it is believed that GM-CSF only enhances the macrophage transformation into dendritic cells on the “air side”. The macrophages located in the bronchioles and alveoli express the cytokine IL-2, which is responsible for the recruitment of T cells (CD8+ and CD16+) to the airways. This means that the initially derailed hyperinflammatory state is modified by recruiting T cells from the circulation.

Wong et al. (1985) and Kaushansky et al. (1986) have described the production of recombinant GM-CSF in mammalian cells. Burgess et al. (1987) describes the purification of GM-CSF produced in Escherichia coli.

In one embodiment, GM-CSF according to the present invention is recombinant GM-CSF (rGM-CSF). GM-CSF according to the present invention may be commercially available, e.g. sargramostim (GM-CSF [Leukine®; Immunex, Seattle, Wash.]).

The protein sequence of GM-CSF of Homo Sapiens (SEQ ID NO:1):

### MWLQSLLLLG TVACSISAPA RSPSPSTQPW EHVNAIQEAR RLLNLSRDTA AEMNETVEVI SEMFDLQEPT CLQTRLELYK QGLRGSLTKL KGPLTMMASH YKQHCPPTPE TSCATQIITF ESFKENLKDF LLVIPFDCWE PVQE

### Functional Homologues

A functional homologue of GM-CSF is a polypeptide having at least 50% sequence identity with SEQ ID NO. 1 and has one or more GM-CSF functions, such as the stimulation of the growth and differentiation of hematopoietic precursor cells from various lineages, including granulocytes, macrophages, eosinophils and erythrocytes.

GM-CSF regulates multiple functions of alveolar macrophages (AMs). GM-CSF stimulation of AMs has been documented to enhance their selective response to noxious ingestants, i.e., stimulation of inflammation during bacterial phagocytosis, while the responses to non-noxious ingestants are generally mollified, i.e., anti-inflammatory responses during phagocytosis of apoptotic cells. Further AM functions are enhanced by GM-CSF stimulation with subsequent proliferation, differentiation, accumulation and activation. In addition, these GM-CSF effects also encompass cell adhesion, improved chemotaxis, Fc-receptor expression, complement- and antibody-mediated phagocytosis, oxidative metabolism, intracellular killing of bacteria, fungi, protozoa, and viruses, cytokine signaling, and antigen presentation. GM-CSF also enhances AM cell adhesion, pathogen-associated molecular-pattern receptors, like Toll-like receptors and TLR trans-membranous signaling, surfactant protein and lipid uptake and degradation (Trapnell and Whitsett, 2002).

Further, GM-CSF interacts with the AM's recognition receptors, the so-called Toll-like receptors (TLR). GM-CSF is important for the pulmonary host defense in pneumonia because of its interaction with the TLRs' participation in host defense, resulting in enhanced clearance of the causative microorganism (Chen et al., 2007). The lung has its own innate GM-CSF production, which is reduced in pneumonia and hyperoxia in relation to high O₂ exposure, as seen in e.g. ventilator-associated pneumonia (VAP), contributing to the impairment of host defense secondary to apoptosis with poor response to infections. The hyperoxic injury seems to be counteracted by activation of AMs by GM-CSF (Altemeier et al., 2007; Baleeiro et al., 2006) with subsequent clearance of P. aeruginosa via expression of the TLR signaling pathway (Baleeiro et al., 2006).

Finally, GM-CSF stimulates the in-vitro conversion of AMs into immature dendritic cells (DCs), which may be further matured by means of specific agents with respect to activating the homing of matured DCs to a specific receptor or target (Zobywalski et al., 2007).

Preferably, the evolutionary conservation of amino-acid residues in GM-CSFs of different closely related species, e.g. as assessed by sequence alignment, can be used to pinpoint the degree of evolutionary pressure on individual amino-acid residues. Preferably, GM-CSF amino-acid sequences are compared between species where GM-CSF function is conserved, for example, but not limited to, mammals including rodents and non-human primates (monkeys and apes). Residues that remain constant between species or are under high selective pressure are more likely to represent essential amino acids that cannot easily be substituted than residues that change between species. It is evident from the above that a reasonable number of modifications or alterations of the human GM-CSF sequence does not interfere with the activity of the GM-CSF molecule according to the invention. Such GM-CSF molecules are herein referred to as functional equivalents of human GM-CSF, and may be such as variants and fragments of native human GM-CSF as described here below.

As used herein, the expression “variant” refers to polypeptides or proteins which are homologous to the index protein, which is suitably human GM-CSF, but which differ from the index sequence from which they are derived in that one or more amino acids within the sequence are substituted by other amino acids. Amino acid substitutions may be regarded as “conservative” where an amino acid is replaced with a different amino acid with broadly similar properties. Non-conservative substitutions are where amino acids are replaced with amino acids of a different type. Broadly speaking, fewer non-conservative substitutions will be possible without altering the biological activity of the polypeptide.

A person skilled in the art will know how to make and assess ‘conservative’ amino acid substitutions, by which one amino acid is substituted for another with one or more shared chemical and/or physical characteristics. Conservative amino acid substitutions are less likely to affect the functionality of the protein. Amino acids may be grouped according to shared characteristics. A conservative amino acid substitution is a substitution of one amino acid within a predetermined group of amino acids for another amino acid within the same group, wherein the amino acids within a predetermined groups exhibit similar or substantially similar characteristics. Within the meaning of the term “conservative amino acid substitution” as applied herein, one amino acid may be substituted for another within groups of amino acids characterized by having
i) polar side chains (Asp, Glu, Lys, Arg, His, Asn, Gln, Ser, Thr, Tyr, and Cys,)
ii) non-polar side chains (Gly, Ala, Val, Leu, Ile, Phe, Trp, Pro, and Met)
iii) aliphatic side chains (Gly, Ala Val, Leu, Ile)
iv) cyclic side chains (Phe, Tyr, Trp, His, Pro)
v) aromatic side chains (Phe, Tyr, Trp)
vi) acidic side chains (Asp, Glu)
vii) basic side chains (Lys, Arg, His)
viii) amide side chains (Asn, Gln
ix) hydroxy side chains (Ser, Thr)
x) sulfur-containing side chains (Cys, Met), and
xi) amino acids being monoamino-dicarboxylic acids (Asp, Glu) or monoamino-monocarboxylic-monoamidocarboxylic acids, (Asn, Gln).

A functional homologue within the scope of the present invention is a polypeptide that exhibits at least 50% sequence identity with human GM-CSF as identified by SEQ ID NO. 1, preferably at least 60% sequence identity, for example 70% sequence identity, and preferably functional homologues have at least 75% sequence identity, for example at least 80% sequence identity, such as at least 85% sequence identity, for example at least 90% sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with SEQ ID NO: 1.

Sequence identity can be calculated using a number of well-known algorithms and applying a number of different gap penalties. Any sequence alignment algorithm, such as but not limited to FASTA, BLAST, or GETSEQ can be used for searching for homologues and calculating sequence identity. Moreover, when appropriate, any commonly known substitution matrix, such as but not limited to PAM, BLOSSUM or PSSM matrices, may be applied with the search algorithm. For example, a PSSM (position-specific scoring matrix) may be applied via the PSI-BLAST program. Moreover, sequence alignments may be performed using a range of penalties for gap opening and extension. For example, the BLAST algorithm may be used with a gap opening penalty in the range 5-12, and a gap extension penalty in the range 1-2.

Accordingly, a variant or a fragment thereof according to the invention may comprise, within the same variant of the sequence or fragments thereof or among different variants of the sequence or fragments thereof, at least one substitution, such as a plurality of substitutions introduced independently of one another.

It is clear from the above outline that the same variant or fragment thereof may comprise more than one conservative amino-acid substitution from more than one group of conservative amino acids as defined herein above.

Aside from the twenty standard amino acids and two special amino acids, selenocysteine and pyrrolysine, there are a vast number of “nonstandard amino acids” which are not incorporated into protein in vivo. Examples of nonstandard amino acids include the sulfur-containing taurine, the neurotransmitter gamma-amino butyric acid (GABA) and the neurotransmitter precursor dihydroxyphenylalanine (DOPA). Other examples are lanthionine, 2-aminoisobutyric acid, and dehydroalanine. Further nonstandard amino acids are ornithine and citrulline.

Further examples of nonstandard amino acids include hydroxylated amino acids such as hydroxyproline and hydroxylysine.

Non-standard amino acids are usually formed through modifications to standard amino acids. For example, taurine can be formed by the decarboxylation of cysteine, while DOPA is synthesized from tyrosine and hydroxyproline is made by a posttranslational modification of proline (common in collagen). Examples of non-natural amino acids are those listed e.g. in 37 C.F.R. section 1.822(b)(4), all of which are incorporated herein by reference.

Both standard and nonstandard amino acid residues described herein can be in the “D” or “L” isomeric form.

It is contemplated that a functional equivalent according to the invention may comprise the inclusion or substitution in the sequence of any amino acid including nonstandard amino acids. In preferred embodiments a functional equivalent comprises only standard amino acids.

The standard and/or nonstandard amino acids may be linked by peptide bonds or by non-peptide bonds. The term peptide also embraces post-translational modifications introduced by chemical or enzyme-catalyzed reactions, as are known in the art. Such post-translational modifications can be introduced prior to partitioning, if desired. Amino acids as specified herein will preferentially be in the L-stereoisomeric form. Amino-acid analogs can be employed instead of the 20 naturally-occurring amino acids. Several such analogs are known, including fluorophenylalanine, norleucine, azetidine-2-carboxylic acid, S-aminoethyl cysteine, 4-methyl tryptophan and the like.

Suitable variants will have at least 60% sequence identity, for example 70% sequence identity, and variants will preferably have at least 75% sequence identity, for example at least 80% sequence identity, such as at least 85% sequence identity, for example at least 90% sequence identity, such as at least 91% sequence identity, for example at least 92% sequence identity, such as at least 93% sequence identity, for example at least 94% sequence identity, such as at least 95% sequence identity, for example at least 96% sequence identity, such as at least 97% sequence identity, for example at least 98% sequence identity, such as at least 99% sequence identity with the predetermined sequence of human GM-CSF.

Functional equivalents may further comprise chemical modifications such as ubiquitination, labeling (e.g., with radionuclides, various enzymes, etc.), pegylation (derivatization with polyethylene glycol), or by insertion (or substitution by chemical synthesis) of amino acids (amino acids) such as ornithine, which do not normally occur in human proteins.

In addition to the peptidyl compounds described herein, sterically similar compounds may be formulated to mimic the key portions of the peptide structure and that such compounds may also be used in the same manner as the peptides of the invention. This may be achieved by techniques of molecular modeling and chemical designing known to those of skill in the art. For example, esterification and other alkylations may be employed to modify the amino terminus of, e.g., a di-arginine peptide backbone, to mimic a tetrapeptide structure. It will be understood that all such sterically similar constructs fall within the scope of the present invention.

Peptides with N-terminal alkylations and C-terminal esterifications are also encompassed within the present invention. Functional equivalents also comprise glycosylated and covalent or aggregative conjugates formed with the same molecules, including dimers or complexes with unrelated chemical moieties. Such functional equivalents are prepared by linkage of functionalities to groups which are found in fragment including at any one or both of the N- and C-termini, by means known in the art.

Functional homologues may further include peptides with N-terminal carboxylation and/or C-terminal amidation. Thus in one embodiment of the present invention, a functional homologue may comprise a peptide having one or more N-terminal modifications selected from the list including alkylations and carboxylations, and/or a peptide having one or more C-terminal modifications selected from the list including esterifications and amidations.

In a particular embodiment of the present invention, functional homologues also comprise glycosylated and covalent or aggregative conjugates which are homodimers.

The term “fragment thereof” may refer to any portion of the given amino-acid sequence. Fragments may comprise more than one portion from within the full-length protein, joined together. Suitable fragments may be deletion or addition mutants. The addition of at least one amino acid may be an addition of from preferably 2 to 250 amino acids, such as from 10 to 20 amino acids, for example from 20 to 30 amino acids, such as from 40 to 50 amino acids. Fragments may include small regions from the protein or combinations of these.

Suitable fragments may be deletion or addition mutants. The addition or deletion of at least one amino acid may be an addition or deletion of from preferably 2 to 250 amino acids, such as from 10 to 20 amino acids, for example from 20 to 30 amino acids, such as from 40 to 50 amino acids. The deletion and/or the addition may, independently of one another, be a deletion and/or an addition within a sequence and/or at the end of a sequence.

Deletion mutants suitably comprise at least 20 or 40 consecutive amino acids and more preferably at least 80 or 100 consecutive amino acids in length. Accordingly such a fragment may be a shorter sequence of the sequence as identified by SEQ ID NO: 1 comprising at least 20 consecutive amino acids, for example at least 30 consecutive amino acids, such as at least 40 consecutive amino acids, for example at least 50 consecutive amino acids, such as at least 60 consecutive amino acids, for example at least 70 consecutive amino acids, such as at least 80 consecutive amino acids, for example at least 90 consecutive amino acids, such as at least 95 consecutive amino acids, such as at least 100 consecutive amino acids, such as at least 105 amino acids, for example at least 110 consecutive amino acids, such as at least 115 consecutive amino acids, for example at least 120 consecutive amino acids, wherein said deletion mutants preferably has at least 75% sequence identity, for example at least 80% sequence identity, such as at least 85% sequence identity, for example at least 90% sequence identity, such as at least 91% sequence identity, for example at least 92% sequence identity, such as at least 93% sequence identity, for example at least 94% sequence identity, such as at least 95% sequence identity, for example at least 96% sequence identity, such as at least 97% sequence identity, for example at least 98% sequence identity, such as at least 99% sequence identity with SEQ ID NO: 1.

It is preferred that functional homologues of GM-CSF comprises at most 500, more preferably at most 400, even more preferably at most 300, yet more preferably at most 200, such as at most 175, for example at most 160, such as at most 150 amino acids, for example at most 144 amino acids.

The term “fragment thereof” may refer to any portion of the given amino acid sequence. Fragments may comprise more than one portion from within the full-length protein, joined together. Portions will suitably comprise at least 5 and preferably at least 10 consecutive amino acids from the basic sequence. They may include small regions from the protein or combinations of these.

There are two known variants of human GM-CSF; a T115I substitution in variant 1 and an I117T substitution in variant 2. Accordingly, in one embodiment of the invention functional homologues of GM-CSF comprises a sequence with high sequence identity to SEQ ID NO: 1 or any of the splice variants.

Analogs of GM-CSF are for example described in U.S. Pat. Nos. 5,229,496, 5,393,870, and 5,391,485 by Deeley et al. Such analogues are also functional equivalents comprised within the present invention.

In one embodiment GM-CSF is used according to the present invention in homo- or heteromeric form. Homo- and heteromeric forms of GM-CSF may comprise one or more GM-CSF monomers or functional homologous of GM-CSF as defined herein above. Homo- and heteromers include dimers, trimers, tetramers, pentamers, hexamers, heptamers, octamers, nonamers and decamers.

In one embodiment, a homodimer, trimer or tetramer of GM-CSF is used.

### Recombinant Production

The present invention relates to the pulmonary administration of GM-CSF, or a functional homologue thereof, however prepared, to treat bronchial asthma in a patient in need thereof. GM-CSF can be produced in various ways, such as isolation from for example human or animal serum or from expression in cells, such as prokaryotic cells, yeast cells, insect cells, mammalian cells or in cell-free systems.

In one embodiment of the invention, GM-CSF is produced recombinantly by host cells.

Thus, in one aspect of the present invention, GM-CSF is produced by host cells comprising a first nucleic acid sequence encoding the GM-CSF operably associated with a second nucleic acid capable of directing expression in said host cells. The second nucleic acid sequence may thus comprise or even consist of a promoter that will direct the expression of protein of interest in said cells. A skilled person will be readily capable of identifying useful second nucleic acid sequence for use in a given host cell.

The process of producing recombinant GM-CSF in general comprises the steps of:
providing a host cell,
preparing a gene expression construct comprising a first nucleic acid encoding GM-CSF operably linked to a second nucleic acid capable of directing expression of said protein of interest in the host cell,
transforming the host cell with the construct,
cultivating the host cell, thereby obtaining expression of GM-CSF.

The recombinant GM-CSF thus produced may be isolated by any conventional method, such as any of the methods for protein isolation described herein below. The skilled person will be able to identify a suitable protein isolation steps for purifying GM-CSF.

In one embodiment of the invention, the recombinantly produced GM-CSF is excreted by the host cells. When GM-CSF is excreted the process of producing a recombinant protein of interest may comprise the following steps:
providing a host cell,
preparing a gene expression construct comprising a first nucleic acid encoding GM-CSF operably linked to a second nucleic acid capable of directing expression of said protein of interest in said host cell,
transforming said host cell with the construct,
cultivating the host cell, thereby obtaining expression of GM-CSF and secretion of GM-CSF into the culture medium,
hereby obtaining culture medium comprising GM-CSF.

The composition comprising GM-CSF and nucleic acids may thus in this embodiment of the invention be the culture medium or a composition prepared from the culture medium.

In another embodiment of the invention said composition is an extract prepared from animals, parts thereof or cells or an isolated fraction of such an extract.

In an embodiment of the invention, GM-CSF is recombinantly produced in vitro in host cells and is isolated from cell lysate, cell extract or from tissue culture supernatant. In a more preferred embodiment GM-CSF is produced by host cells that are modified in such a way that they express GM-CSF. In an even more preferred embodiment of the invention said host cells are transformed to produce and excrete GM-CSF.

### Administration

Administration of an effective amount of GM-CSF or a functional homologue thereof via e.g. intratracheal, intrabronchial or bronchoalveolar administration is particularly useful in alleviating symptoms and/or treating subjects suffering from bronchial asthma, particularly severe bronchial asthma. The administration or treatment may either be prophylactic or therapeutic.

Thus, according to the present invention, an effective amount of GM-CSF or a functional homologue thereof is administered via pulmonary administration, such as by inhalation or intratracheal, intrabronchial or intraalveolar administration.

Inhalation of an effective amount of GM-CSF or a functional homologue thereof, such as for example via a nebulized solution, or via a powder form, is useful in alleviating symptoms or signs of bronchial asthma and/or treating subjects suffering from bronchial asthma of various forms.

In one embodiment, GM-CSF is administered systemically, preferably subcutaneously.

Intravenous administration of GM-CSF is not recommended as GM-CSF is rapidly metabolized and cleared from the circulation.

GM-CSF according to the present invention may be administered in any suitable way or form to achieve an effect on bronchial asthma, preferably by pulmonary administration including intratracheal, intrabronchial or bronchoalveolar administration. In one embodiment of the present invention, the GM-CSF or a functional equivalent thereof is administered via inhalation such as by inhalation of a nebulized solution or powder comprising GM-CSF or a functional homologue thereof.

In severe forms of bronchial asthma with partial or complete obstruction of the airways, systemic administration of GM-CSF is also recommended.

Methods of intratracheal, intrabronchial or bronchoalveolar administration include, but are not limited to, spraying, lavage, inhalation, flushing or installation, using as fluid a physiologically acceptable composition in which GM-CSF have been dissolved. When used herein the terms “intratracheal, intrabronchial or intraalveolar administration” include all forms of such administration whereby GM-CSF is applied into the trachea, the bronchi or the alveoli, respectively, whether by the instillation of a solution of GM-CSF, by applying GM-CSF in a powder form, or by allowing GM-CSF to reach the relevant part of the airway by inhalation of GM-CSF as an aerosolized or nebulized solution or suspension or inhaled powder or gel, with or without added stabilizers or other excipients.

Methods of intrabronchial or intraalveolar administration include, but are not limited to, bronchoalveolar lavage (BAL) according to methods well known to those skilled in the art, using as a lavage fluid a physiologically acceptable composition in which GM-CSF been dissolved or indeed by any other effective form of intrabronchial administration including the use of inhaled powders containing GM-CSF in dry form, with or without excipients, or the direct application of GM-CSF, in solution or suspension or powder form during bronchoscopy. Methods for intratracheal administration include, but are not limited to, blind tracheal washing with a similar solution of dissolved GM-CSF or a GM-CSF suspension, or the inhalation of nebulized fluid droplets containing dissolved GM-CSF or a GM-CSF suspension obtained by use of any nebulizing apparatus adequate for this purpose.

In another embodiment, intratracheal, intrabronchial or intraalveolar administration does not include inhalation of the product but the instillation or application of a solution of GM-CSF or a powder or a gel containing GM-CSF into the trachea or lower airways.

Other preferred methods of administration may include using the following devices:
1. Pressurized nebulizers using compressed air/oxygen mixture
2. Ultrasonic nebulizers
3. Electronic micropump nebulizers (e.g. Aeroneb Professional Nebulizer)
4. Metered dose inhaler (MDI)
5. Dry powder inhaler systems (DPI).

The aerosol may be delivered by a) facemasks or b) endotracheal tubes in intubated patients during mechanical ventilation (device 1, 2 and 3). The devices 4 and 5 can also be used by the patient without assistance, provided that the patient is able to self-activate the aerosol device.

The treatment with GM-CSF or a functional homologue thereof is particularly effective when GM-CSF or a functional homologue thereof reaches the parts of the airways where the macrophages are protecting the airways against incoming (inhaled) antigens such as the small airways.

The small airways at the level of the bronchiole are specifically vulnerable with respect to the allergic response, because of the air-blood barrier, which separates the air compartment in the airway lumen from the blood compartment. When asthma becomes irreversible due to allergic or asthmatic bronchiolitis, the lumen of these airways may be completely blocked by edema fluid, edema of the airway wall, constriction of the airway smooth muscle, rejected mucosa cells and cellular debris, all consequences of the inflammatory process involving the activation of eosinophilic granulocytes.

In a case of severe asthmatic bronchiolitis, a severe form of bronchiolitis, the bronchioles may be blocked so as to hinder the penetration of an inhaled GM-CSF aerosol. Adequate alleviation of the condition will require delivery of GM-CSF to the target site both above and preferably below the site of blockage to the passage of aerosol.

Improved penetration of inhaled GM-CSF to its target site, the small airways (bronchioles and alveoli) may be obtained by:
(i) Earlier intervention, e.g. already in the early phase of acute severe asthma, i.e. when the patient no longer respond to β₂-agonists, or when the asthmatic condition becomes unresponsive to maximal anti-asthmatic therapy including short- and long-acting bronchodilators and corticosteroids administered both orally and by inhalation.
(ii) Giving a higher dose-rate of inhaled GM-CSF in order to achieve the wanted effect.
(iii) Applying continuous positive airway pressure (CPAP) with spontaneous breathing or extrinsic positive end-expiratory pressure (PEEP; mechanical ventilation) in order to facilitate the delivery of inhaled GM-CSF to the distal airways and enhance its effect.

### Collateral Ventilation and Drug Delivery

The two applications of increased airway pressure (CPAP and PEEP) may increase the collateral ventilation (CV) (Menkes et al 1979), so to say “from behind” via the ventilation pores between the terminal units of peripheral airways. The phenomenon of CV can be particularly useful in pulmonary disease with anatomical partial or total block of the airways, since it can increase delivery of drugs to the site of interest which is the peripheral airways.

By exploiting the occurrence of CV, CPAP and PEEP may cause air to bypass obstructed airways through collateral channels including interalveolar pores, bronchiole-alveolar communications, and interbronchiolar pathways. Resistance through these channels located at the small airways increases with decreasing lung volume.

Functional blockage of the airways to the passage of an aerosol may thus be alleviated exploiting the occurrence of CV, which facilitates the distribution of GM-CSF or a functional homologue thereof into the airways beyond the level of obstruction. Stages of evolving or partial blockage may also be successfully treated by this method. Thus, in one embodiment of the present invention, inhaled GM-CSF or a functional homologue thereof is administered via inhalation combined with collateral ventilation, such as CPAP and/or PEEP.

Preferred concentrations for a solution comprising GM-CSF and/or functional homologues or variants of GM-CSF are in the range of 0.1 μg to 10000 μg active ingredient per mL of solution. The suitable concentrations are often in the range of from 0.1 μg to 5000 μg per mL of solution, such as in the range of from about 0.1 μg to 3000 μg per mL of solution, and especially in the range of from about 0.1 μg to 1000 μg per mL of solution, such as in the range of from about 0.1 μg to 250 μg per mL solution. A preferred concentration would be from about 0.1 to about 5.0 mg, preferably from about 0.3 mg to about 3.0 mg, such as from about 0.5 to about 1.5 mg and especially in the range from 0.8 to 1.0 mg per mL of solution.

In one embodiment, GM-CSF is administered systemically, e.g. by subcutaneous injection.

In one embodiment, the GM-CSF is used to treat a mammal, such as a human subject. The human subject may be a child of less than 12 years or an adult older than 12 years.

### Pharmaceutical Composition

Pharmaceutical compositions or formulations for use in the present invention include GM-CSF or functional homologue thereof combination with, preferably dissolved in, a pharmaceutically acceptable carrier, preferably an aqueous carrier or diluent, or carried to the lower airways as a pegylated preparation or as a liposomal or nanoparticle preparation administered as an aerosol via inhalation, or as a lavage fluid administered via a bronchoscope as a bronchoalveolar lavage or as a blind intratracheal wash or lavage. A variety of aqueous carriers may be used, including, but not limited to 0.9% saline, buffered saline, physiologically compatible buffers and the like. The compositions may be sterilized by conventional techniques well known to those skilled in the art. The resulting aqueous solutions may be packaged for use or filtered under aseptic conditions and freeze-dried, the freeze-dried preparation being dissolved in a sterile aqueous solution prior to administration

In one embodiment a freeze-dried GM-CSF preparation may be pre-packaged for example in single dose units. In an even more preferred embodiment the single dose unit is adjusted to the patient.

The compositions may contain pharmaceutically acceptable auxiliary substances or adjuvants, including, without limitation, pH adjusting and buffering agents and/or tonicity adjusting agents, such as, for example, sodium acetate, sodium lactate, sodium chloride, potassium chloride, calcium chloride, etc.

The formulations may contain pharmaceutically acceptable carriers and excipients including microspheres, liposomes, microcapsules, nanoparticles or the like. Conventional liposomes are typically composed of phospholipids (neutral or negatively charged) and/or cholesterol. The liposomes are vesicular structures based on lipid bilayers surrounding aqueous compartments. They can vary in their physiochemical properties such as size, lipid composition, surface charge and number and fluidity of the phospholipids bilayers. The most frequently used lipid for liposome formation are: 1,2-Dilauroyl-sn-Glycero-3-Phosphocholine (DLPC), 1,2-Dimyristoyl-sn-Glycero-3-Phosphocholine (DMPC), 1,2-Dipalmitoyl-sn-Glycero-3-Phosphocholine (DPPC), 1,2-Distearoyl-sn-Glycero-3-Phosphocholine (DSPC), 1,2-Dioleoyl-sn-Glycero-3-Phosphocholine (DOPC), 1,2-Dimyristoyl-sn-Glycero-3-Phosphoethanolamine (DMPE), 1,2-Dipalmitoyl-sn-Glycero-3-Phosphoethanolamine (DPPE), 1,2-Dioleoyl-sn-Glycero-3-Phosphoethanolamine (DOPE), 1,2-Dimyristoyl-sn-Glycero-3-Phosphate (Monosodium Salt) (DMPA), 1,2-Dipalmitoyl-sn-Glycero-3-Phosphate (Monosodium Salt) (DPPA), 1,2-Dioleoyl-sn-Glycero-3-Phosphate (Monosodium Salt) (DOPA), 1,2-Dimyristoyl-sn-Glycero-3-[Phospho-rac-(1-glycerol)] (Sodium Salt) (DMPG), 1,2-Dipalmitoyl-sn-Glycero-3-[Phospho-rac-(1-glycerol)] (Sodium Salt) (DPPG), 1,2-Dioleoyl-sn-Glycero-3-[Phospho-rac-(1-glycerol)] (Sodium Salt) (DOPG), 1,2-Dimyristoyl-sn-Glycero-3-[Phospho-L-Serine] (Sodium Salt) (DMPS), 1,2-Dipalmitoyl-sn-Glycero-3-[Phospho-L-Serine) (Sodium Salt) (DPPS), 1,2-Dioleoyl-sn-Glycero-3-[Phospho-L-Serine] (Sodium Salt) (DOPS), 1,2-Dioleoyl-sn-Glycero-3-Phosphoethanolamine-N-(glutaryl) (Sodium Salt) and 1,1′,2,2′-Tetramyristoyl Cardiolipin (Ammonium Salt). Formulations composed of DPPC in combination with other lipids or modifiers of liposomes are preferred e.g. in combination with cholesterol and/or phosphatidylcholine.

Long-circulating liposomes are characterized by their ability to extravasate at body sites where the permeability of the vascular wall is increased. The most popular way of producing long-circulating liposomes is to attach hydrophilic polymer polyethylene glycol (PEG) covalently to the outer surface of the liposome. Some of the preferred lipids are: 1,2-Dipalmitoyl-sn-Glycero-3-Phosphoethanolamine-N-[Methoxy(Polyethylene glycol)-2000] (Ammonium Salt), 1,2-Dipalmitoyl-sn-Glycero-3-Phosphoethanolamine-N-[Methoxy(Polyethylene glycol)-5000] (Ammonium Salt), 1,2-Dioleoyl-3-Trimethylammonium-Propane (Chloride Salt) (DOTAP).

Possible lipids applicable for liposomes are supplied by Avanti, Polar Lipids, Inc., Alabaster, Ala. Additionally, the liposome suspension may include lipid-protective agents which protect lipids against free-radical and lipid-peroxidative damage on storage. Lipophilic free-radical quenchers, such as alpha-tocopherol and water-soluble iron-specific chelators, such as ferrioxamine, are preferred.

A variety of methods are available for preparing liposomes, as described in e.g. Szoka et al. (1980), and in U.S. Pat. Nos. 4,235,871, 4,501,728 and 4,837,028, all of which are incorporated herein by reference. Another method produces multi-lamellar vesicles of heterogeneous sizes. In this method, the vesicle-forming lipids are dissolved in a suitable organic solvent or solvent system and dried under vacuum or an inert gas to form a thin lipid film. If desired, the film may be re-dissolved in a suitable solvent, such as tertiary butanol, and then lyophilized to form a more homogeneous lipid mixture which is in a more easily hydrated powder-like form. This film is covered with an aqueous solution of the targeted drug and the targeting component and allowed to hydrate, typically over a 15-60 minute period with agitation. The size distribution of the resulting multi-lamellar vesicles can be shifted toward smaller sizes by hydrating the lipids under more vigorous agitation conditions or by adding solubilizing detergents such as deoxycholate.

Micelles are formed by surfactants (molecules that contain a hydrophobic portion and one or more ionic or otherwise strongly hydrophilic groups) in aqueous solution.

Common surfactants well known to one of skill in the art can be used in the micelles of the present invention. Suitable surfactants include sodium laureate, sodium oleate, sodium lauryl sulfate, octaoxyethylene glycol monododecyl ether, octoxynol 9 and PLURONIC F-127 (Wyandotte Chemicals Corp.). Preferred surfactants are nonionic polyoxyethylene and polyoxypropylene detergents compatible with IV injection such as, TWEEN-80, PLURONIC F-68, n-octyl-beta-D-glucopyranoside, and the like. In addition, phospholipids, such as those described for use in the production of liposomes, may also be used for micelle formation.

In some cases, it will be advantageous to include a compound, which promotes delivery of the active substance to its target.

### Dose

By “effective amount” of GM-CSF it is meant a dose, which, when administered to a patient in need thereof, e.g. by pulmonary administration, achieves a concentration in the subject's airways and/or lung parenchyma which has a beneficial effect on bronchial asthma, i.e. by alleviating and/or preventing asthma symptoms.

The preparations are administered in a manner compatible with the dosage formulation, and in such amount as will be therapeutically effective. The quantity to be administered depends on the subject to be treated, including, e.g. the weight and age of the subject, the disease to be treated and the stage of disease. Suitable dosage ranges are per kilogram of body weight normally of the order of several hundred μg active ingredient per administration with a preferred range of from about 0.1 μg to 10000 μg per kilogram of body weight. Doses expected to provide an effective amount of GM-CSF comprise GM-CSF are often in the range of from 0.1 μg to 5000 μg per kilogram of body weight, such as in the range of from about 0.1 μg to 3000 μg per kilogram of body weight, and especially in the range of from about 0.1 μg to 1000 μg per kilogram of body weight, preferably in the range of 5 μg to 1000 μg, even more preferred about 100 μg to about 800 μg administered via inhalation once, twice or three times daily.

Suitable daily dosage ranges are per kilogram of body weight per day normally of the order of several hundred μg active ingredient per day with a preferred range of from about 0.1 μg to 10000 μg per kilogram of body weight per day. Using monomeric forms of the compounds, the suitable dosages are often in the range of from 0.1 μg to 5000 μg per kilogram of body weight per day, such as in the range of from about 0.1 μg to 3000 μg per kilogram of body weight per day, and especially in the range of from about 0.1 μg to 1000 μg per kilogram of body weight per day, when based on monomeric forms having a sequence identical to sequence ID NO: 1, for functional homologues and fragments the dose is calculated according to the molecular weight of the monomeric form to the molecular weight of the homologues or fragments.

The dose of functional homologues and fragments is further calculated according to the ratio of their biological activity to that of the parent compound.

GM-CSF may e.g. be administered by inhalation to a patient suffering from moderate to severe asthma in a dose ranging from about 300 μg administered once a day to about 600 μg administered three times a day.

Patients suffering from severe forms of bronchial asthma characterized by clogging of the airways may particularly benefit from GM-CSF administered via inhalation and systemic treatment with GM-CSF, such as by administration of about 300 μg GM-CSF subcutaneously.

Hence, in one embodiment, GM-CSF is administered to a patient suffering from severe bronchial asthma and in risk of developing acute severe bronchial asthma.

Duration of dosing will typically range from 1 day to about 4 months, such as 2 days to about 3 months, for example in the range of 1 or 2 days to 2 months, such as in the range of 1 or 2 days to 1 month.

For example a duration of dosing may be in the range of 1 to 14 days, such as 2 to 3 days, or 3 days to 4 days, or 4 to 5 days, such as in the range of 5 to 14 days, such as 5 to 6 days, or 6 to 7 days, or 7 to 14 days, such as one week to two weeks, for example two to four weeks, such as one month to two months, for example 2 to 4 months, or for as long as symptoms and signs of disease are detectable. An intervention of at least 10 days is preferred.

The transformation of a resting macrophage into a fully immunocompetent dendritic cell after in-vitro incubation of macrophages with GM-CSF takes approximately 10 days.

In a preferred embodiment of the invention, the duration of dosing has the length to allow for said transformation. Thus the duration can be in the range of 5 to 14 days, for example 5 days, or for example 6 days, or for example 13 days, or for example 14 days, even more preferably in the range of 7 to 12 days such as for example 7 days, or for example 8 days, or for example 9 days, or for example 10 days, or for example 11 days, or for example 12 days.

A dosage regimen may alternate between periods of administration of the pharmaceutical composition according to the present invention and periods without such administration (a pause in treatment). A pause in treatment in such a dosage regimen may last 5 to 10 days, for example 5 days, or for example 6 days, or for example 7 days, or for example 8 days, or for example 9 days, or for example 10 days or more, for example 10 days to 4 months or until symptoms or signs of bronchial asthma are observed.

It has already been shown that increasing the dose of inhaled GM-CSF produces an enhanced survival in animal models. In as much as inhaled GM-CSF has not been found to be toxic even in supra-physiological doses, an increased dose rate may further counteract the deleterious effect of the pre-existing severe asthmatic condition.

Examples of dosage regimens may include a cycle of 10 days of treatment with the pharmaceutical composition according to the present invention and 7 days of pause in treatment. In one embodiment, the GM-CSF according to the present invention is administered to a patient in need thereof whenever there is a need to alleviate or prevent symptoms of bronchial asthma.

### Medical Packaging

The compounds used in the invention may be administered alone or in combination with pharmaceutically acceptable carriers or excipients, in either single or multiple doses. The formulations may conveniently be presented in unit dosage form by methods known to those skilled in the art.

It is preferred that the compounds according to the invention are provided in a kit. Such a kit typically contains an active compound in dosage forms for administration. A dosage form contains a sufficient amount of active compound such that a desirable effect can be obtained when administered to a subject.

Thus, it is preferred that the medical packaging comprises an amount of dosage units corresponding to the relevant dosage regimen. Accordingly, in one embodiment, the medical packaging comprises a pharmaceutical composition comprising a compound as defined above or a pharmaceutically acceptable salt thereof and pharmaceutically acceptable carriers, vehicles and/or excipients, said packaging comprising from 1 to 7 dosage units, thereby having dosage units for one or more days, or from 7 to 21 dosage units, or multiples thereof, thereby having dosage units for one week of administration or several weeks of administration.

The dosage units can be as defined above. The medical packaging may be in any suitable form for intratracheal, intrabronchial or intraalveolar administration. In a preferred embodiment the packaging is in the form of a vial, ampule, tube, blister pack, cartridge or capsule.

When the medical packaging comprises more than one dosage unit, it is preferred that the medical packaging is provided with a mechanism to adjust each administration to one dosage unit only.

Preferably, a kit contains instructions indicating the use of the dosage form to achieve a desirable affect and the amount of dosage form to be taken over a specified time period. Accordingly, in one embodiment the medical packaging comprises instructions for administering the pharmaceutical composition.

Even more preferably a freeze-dried GM-CSF preparation may be pre-packaged for example in single dose units. In an even more preferred embodiment the single dose unit is adjusted to the patient.

### EXAMPLES

### Example 1

### Severe Acute Bronchial Asthma

Clinical Case:

A 45-year-old man with a history of bronchial asthma since childhood experienced increasing dyspnea over 4 days. Day 0 is the day of hospitalization.

Day 16: The patient experienced increasing dyspnea and wheezing, with decreasing effect of even large doses of prednisolone. Over the next three days he experienced constant severe dyspnea, even when sitting in an upright position in order to sleep.

Day 19: The patient started inhalation of GM-CSF at a dose of 300 μg a day via a micropump nebulizer.

Day 25: The condition was stabilized with a clear decrease in shortness of breath.

Day 35: A productive cough started and the patients' symptoms improved markedly, with reversal of the decline in lung function.

### REFERENCES

### Non-Patent Literature

Altemeier W A, Zhu X, Berrington W R, Harlan J M, Liles W C (2007) Fas (CD95) induces macrophage proinflammatory chemokine production via a MyD88-dependent, caspase-independent pathway. J Leukoc Biol 82:721-728.
Baleeiro C E, Christensen P J, Morris S B, Mendez M P, Wilcoxen S E, Paine R 3rd (2006) GM-CSF and the impaired pulmonary innate immune response following hyperoxic stress. Am J Physiol Lung Cell Mol Physiol 291:L1246-L1255.
Brown C B, Pihl C E, Kaushansky K (1994) Mapping of human granulocyte-macrophage-colony-stimulating-factor domains interacting with the human granulocyte-macrophage-colony-stimulating-factor-receptor alpha-subunit. Eur J Biochem 225:873-880.
Burgess A W, Begley C G, Johnson G R, Lopez A F, Williamson D J, Mermod J J, Simpson R J, Schmitz A, DeLamarter J F (1987) Purification and properties of bacterially synthesized human granulocyte-macrophage colony stimulating factor. Blood 69:43-51.
Cebon J, Nicola N, Ward M, Gardner I, Dempsey P, Layton J, Dührsen U, Burgess A W, Nice E, Morstyn G (1990) Granulocyte-macrophage colony stimulating factor from human lymphocytes. The effect of glycosylation on receptor binding and biological activity. J Biol Chem 265:4483-4491.
Chen G H, Olszewski M A, McDonald R A, Wells J C, Paine R 3rd, Huffnagle G B, Toews G B (2007) Role of granulocyte macrophage colony-stimulating factor in host defense against pulmonary Cryptococcus neoformans infection during murine allergic bronchopulmonary mycosis. Am J Pathol 170:1028-1040.
Clark S C, Kamen R (1987) The human hematopoietic colony-stimulating factors. Science 236:1229-1237.
Diederichs K, Jacques S, Boone T, Karplus P A (1991) Low-resolution structure of recombinant human granulocyte-macrophage colony stimulating factor. J Mol Biol 221:55-60.
Gasson J C, Kaufman S E, Weisbart R H, Tomonaga M, Golde D W (1986) High-affinity binding of granulocyte-macrophage colony-stimulating factor to normal and leukemic human myeloid cells. Proc Natl Acad Sci USA 83:669-673.
Gasson J C, Fraser J K, Nimer S D (1990a) Human granulocyte-macrophage colony-stimulating factor (GM-CSF): regulation of expression. Prog Clin Biol Res 338:27-41.
Gasson J C, Baldwin G C, Sakamoto K M, DiPersio J F (1990b) The biology of human granulocyte-macrophage colony-stimulating factor (GM-CSF). Prog Clin Biol Res 352:375-384.
Gearing D P, King J A, Gough N M, Nicola N A (1989) Expression cloning of a receptor for human granulocyte-macrophage colony-stimulating factor. EMBO J 8:3667-3676.
de Groot R P, Coffer P J, Koenderman L (1998) Regulation of proliferation, differentiation and survival by the IL-3/IL-5/GM-CSF receptor family. Cell Signal 10:619-628.
Hamilton J A (2002) GM-CSF in inflammation and autoimmunity. Trends Immunol 23:403-408.
Hart P H, Whitty G A, Piccoli D S, Hamilton J A (1988) Synergistic activation of human monocytes by granulocyte-macrophage colony-stimulating factor and IFN-gamma. Increased TNF-alpha but not IL-1 activity. J Immunol 141:1516-1521.
Hayashida K, Kitamura T, Gorman D M, Arai K, Yokota T, Miyajima A (1990) Molecular cloning of a second subunit of the receptor for human granulocyte-macrophage colony-stimulating factor (GM-CSF): reconstitution of a high-affinity GM-CSF receptor. Proc Natl Acad Sci USA 87:9655-9659
Kaushansky K, O'Hara P J, Berkner K, Segal G M, Hagen F S, Adamson J W (1986) Genomic cloning, characterization, and multilineage growth-promoting activity of human granulocyte-macrophage colony-stimulating factor. Proc Natl Acad Sci USA 83:3101-3105.
Kitamura T, Hayashida K, Sakamaki K, Yokota T, Arai K, Miyajima A (1991) Reconstitution of functional receptors for human granulocyte/macrophage colony-stimulating factor (GM-CSF): evidence that the protein encoded by the AIC2B cDNA is a subunit of the murine GM-CSF receptor. Proc Natl Acad Sci USA 88:5082-5086.
Lopez A F, Vadas M A, Woodcock J M, Milton S E, Lewis A, Elliott M J, Gillis D, Ireland R, Olwell E, Park L S (1991) Interleukin-5, interleukin-3, and granulocyte-macrophage colony-stimulating factor cross-compete for binding to cell surface receptors on human eosinophils. J Biol Chem 266:24741-24747.
Menkes H, Traystman R, Terry P (1979) Collateral ventilation. Fed Proc 38:22-26
Metcalf D (1986) The molecular biology and functions of the granulocyte-macrophage colony-stimulating factors. Blood 67:257-267.
Rapoport A P, Abboud C N, DiPersio J F (1992) Granulocyte-macrophage colony-stimulating factor (GM-CSF) and granulocyte colony-stimulating factor (G-CSF): receptor biology, signal transduction, and neutrophil activation. Blood Rev 6:43-57.
Sato N, Sakamaki K, Terada N, Arai K, Miyajima A (1993) Signal transduction by the high-affinity GM-CSF receptor: two distinct cytoplasmic regions of the common beta subunit responsible for different signaling. EMBO J 12:4181-4189.
Shanafelt A B, Miyajima A, Kitamura T, Kastelein R A (1991a) The amino-terminal helix of GM-CSF and IL-5 governs high affinity binding to their receptors. EMBO J 10:4105-4112.
Shanafelt A B, Johnson K E, Kastelein R A (1991b) Identification of critical amino acid residues in human and mouse granulocyte-macrophage colony-stimulating factor and their involvement in species specificity. J Biol Chem 266:13804-13810.
Szoka F Jr, Papahadjopoulos D (1980) Comparative properties and methods of preparation of lipid vesicles (liposomes). Annu Rev Biophys Bioeng 9:467-508.
Tazawa R, Nakata K, Inoue Y, Nukiwa T (2006) Granulocyte-macrophage colony-stimulating factor inhalation therapy for patients with idiopathic pulmonary alveolar proteinosis: a pilot study; and long-term treatment with aerosolized granulocyte-macrophage colony-stimulating factor: a case report. Respirology 11 Suppl:S61-S64.
Trapnell B C, Whitsett J A (2002) GM-CSF regulates pulmonary surfactant homeostasis and alveolar macrophage-mediated innate host defense. Annu Rev Physiol 64:775-802.
Wong G G, Witek J S, Temple P A, Wilkens K M, Leary A C, Luxenberg D P, Jones S S, Brown E L, Kay R M, Orr E C, et al (1985) Human GM-CSF: molecular cloning of the complementary DNA and purification of the natural and recombinant proteins. Science 228:810-815.
Zobywalski A, Javorovic M, Frankenberger B, Pohla H, Kremmer E, Bigalke I, Schendel D J (2007) Generation of clinical grade dendritic cells with capacity to produce biologically active IL-12p70. J Transl Med 5:18.

### Patents

U.S. Pat. No. 4,235,871
U.S. Pat. No. 4,501,728
U.S. Pat. No. 4,837,028
U.S. Pat. No. 5,229,496
U.S. Pat. No. 5,391,485
U.S. Pat. No. 5,393,870

1-21. (canceled)

22. A method for alleviating symptoms or treating a subject suffering from bronchial asthma comprising:
administering an effective amount of granulocyte macrophage colony stimulating factor (GM-CSF) or a functional homologue thereof to said subject.

23-41. (canceled)

42. The method of claim 22, wherein said subject is suffering from moderate or severe bronchial asthma.

43. The method of claim 22, wherein the effective amount of GM-CSF or a functional homologue thereof is administered via pulmonary administration, inhalation, intratracheal administration, intrabronchial administration, or intraalveolar administration.

44. The method of claim 22, wherein the subject is administered a solution of GM-CSF or a functional homologue thereof via bronchoalveolar lavage or blind tracheal washing.

45. The method of claim 22, wherein the subject is administered a nebulized solution or a suspension of GM-CSF or a functional homologue thereof.

46. The method of claim 22, wherein the subject is administered a nebulized aerosol or an inhaled powder form of GM-CSF or a functional homologue thereof.

47. The method of claim 22, wherein the subject is administered a pegylated, liposomal or nanoparticle prepared form of GM-CSF or a functional homologue thereof.

48. The method of claim 22, wherein the subject is administered GM-CSF or a functional homologue thereof by direct application of GM-CSF or a functional homologue thereof during bronchoscopy.

49. The method of claim 22, wherein an effective amount of GM-CSF or a functional homologue thereof is administered in doses of 0.1 pg to 1000 pg per kilogram body weight.

50. The method according to claim 22, wherein an effective amount of GM-CSF or a functional homologue thereof is administered in doses of 5 pg to 1000 pg, or 100 pg to 800 pg.

51. The method according to claim 22, wherein the GM-CSF or a functional homologue thereof is administered in doses of about 100 pg to about 800 pg administered via inhalation once, twice or three times daily.

52. The method of claim 22, wherein the dosing of GM-CSF or a functional homologue thereof has a duration of 1 day to about 4 months.

53. The method of claim 22, wherein the dosing of GM-CSF or a functional homologue thereof has a duration of 1 to 14 days, or 7 to 12 days.

54. The method of claim 22, wherein the subject is a mammal.

55. The method of claim 54, wherein the mammal is a human subject.

56. The method of claim 55, wherein the human subject is a child younger than 12 years of age.

57. The method of claim 55, wherein the human subject is an adult older than 12 years of age.
